(19) United States
(12) Patent Application Publication
(10) Pub. No.: US 2002/0143340 A1
(43) Pub. Date: Oct. 3, 2002

(54) Fixing pin of dental tissue regeneration membrane

(75) Inventor: Tadashi Kaneko, Tokyo (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: GC Corporation, Tokyo (JP)

(21) Appl. No.: 10/105,343

(22) Filed: Mar. 26, 2002

(30) Foreign Application Priority Data
Mar. 28, 2001 (JP) 2001-091991

(51) Int. Cl. A61B 17/58

(52) U.S. Cl. 606/72

(57) ABSTRACT

To provide a fixing pin, which can be smoothly stuck into a prepared hole bored in a bone without being broken during fixing a dental guided tissue regeneration membrane, and does not readily drop out but can surely fix the dental guided tissue regeneration membrane after being once stuck, the fixing pin made of a bioabsorbable material for fixing a dental guided tissue regeneration membrane is constructed such that a periphery in an oral cavity inner surface side of a head portion thereof supporting a dental guided tissue regeneration membrane is at least in a smooth curve surface form; and that a thumbtack shaft portion thereof to be stuck into a bone is in a columnar having a conical tip and is provided with two to six ridge portions disposed parallel to the axis of the columnar portion thereof and having a length extending within the bone.

### BACKGROUND OF THE INVENTION

1. Field of the Invention

The present invention relates to a fixing pin of a dental tissue regeneration membrane, which, in the remedy of the guided tissue regeneration method to be carried out for the purpose of regenerating a periodontal tissue or regenerating a bone in the dentistry, can surely fix a dental guided tissue regeneration membrane in a prepared hole bored in a bone.

2. Description of the Related Art

In the remedy field such as dentistry and oral surgery, in the case where a cementum, a periodontal ligament, an alveolar bone, and the like are lost, there is employed a remedy by the guided tissue regeneration method, in which for the purpose of regenerating such a periodontal tissue, a dental guided tissue regeneration membrane is implanted, thereby not only securing a space where the periodontal tissue can propagate but also blocking other tissues that likely obstacle the propagation of the periodontal tissue. As the dental guided tissue regeneration membrane, have hitherto been used semi-permeable membranes such as tetrafluoroethylene membranes and polyethylene filters. However, since these materials are non-bioabsorbable, they must be removed after the operation. Accordingly, in recent years, there has been widely employed a remedy by the guided tissue regeneration method, in which as the dental guided tissue regeneration membrane is made of a bioabsorbable polymeric material or collagen, the shape of the membrane is maintained until the periodontal tissue has been propagated and regenerated, and after the remedy, the membrane is rapidly decomposed and absorbed without remaining as a foreign matter within a living body and disappears.

As a method for fixing the dental guided tissue regeneration membrane, employed are a method for binding and fixing it to a tooth with a suture, a method for sandwiching and fixing it between an implant main body and a cover screw, a method for sticking a fixing pin into a bone and fixing it, and other methods. However, according to the method for fixing the dental guided tissue regeneration membrane with a suture, although the side of the dental guided tissue regeneration membrane at which it is bound to the tooth is fixed, the other side becomes unstable. Accordingly, there may possibly occur a phenomenon in which invasion of a gingival connective tissue is caused, or the tissue regeneration is inhibited due to the instability of a wound portion. Further, according to the method for sandwiching and fixing the dental guided tissue regeneration membrane between an implant main body and a cover screw, sure fixing is obtained in the vicinity of the fixed implant, thereby enabling to secure a space. However, there may possibly occur a phenomenon in which the fixing in a periphery of the non-fixed guided tissue regeneration membrane is insufficient so that invasion of a gingival connective tissue is caused, or the tissue regeneration is inhibited due to the instability of a wound portion. On the other hand, according to the method for fixing the dental guided tissue regeneration membrane using a fixing pin, since an operator sticks the fixing pin into a bone of a necessary portion while observing a portion to be operated, sure fixing can be obtained.

As the fixing pin, there have hitherto been used fixing pins made of a metal having biocompatibility, such as titanium, as disclosed in Japanese Patent No. 2,725,989. However, since these fixing pins must be removed after the operation, they cannot make use of the advantage that the dental guided tissue regeneration membrane is bioabsorbable. For this reason, in recent year, the fixing pin has also been made of a bioabsorbable material. In general, its shape is of a thumbtack type as shown in FIG. 3, and the thumbtack shaft is provided with a protrusion of a ring-like shoulder portion for stopping the thumbtack from coming out. And, at the time of the use, there is carried out an operation in which a bone in the site into which the fixing pin is to be stuck is previously bored with a hole having a diameter substantially the same as the diameter of a columnar portion of the thumbtack shaft portion of the fixing pin by means of a drill for preparation, and the fixing pin is then stuck into the prepared hole. However, the ring-like shoulder portion for stopping the thumbtack from coming out substantially increases the diameter of the fixing pin and acts as a resistance during sticking the fixing pin into the hole of the bone. Accordingly, in the case where an excessive force is applied, the ring-like shoulder portion is broken, whereby the coming-out preventing effect is not obtained, and the fixing pin ultimately drops. As the case may be, the fixing pin is often broken at the bottom of the shaft thereof.

### SUMMARY OF THE INVENTION

The present invention is aimed to provide a fixing pin, which during fixing a dental guided tissue regeneration membrane, can be smoothly stuck into a prepared hole bored in a bone without being broken, and after being once stuck, does not readily drop out but can surely fix the dental guided tissue regeneration membrane.

In order to achieve the above-described aim, I, the present inventor made extensive and intensive investigations regarding the shape of a dropping-out preventing portion that is not only able to increase the stiffness of a fixing pin but also to suppress a force acting as a resistance during sticking the fixing pin into a hole as far as possible. As a result, it has been found that it is effective to provide a ridge portion parallel to an axis of a thumbtack shaft portion of the fixing pin, leading to accomplishment of the present invention.

Specifically, the fixing pin of the dental tissue regeneration membrane according to the present invention is a fixing pin of a dental tissue regeneration membrane comprising a bioabsorbable material for fixing a dental guided tissue regeneration membrane, wherein a periphery in an oral cavity inner surface side of a head portion thereof supporting a dental guided tissue regeneration membrane is at least in a smooth curve surface form; and a thumbtack shaft portion thereof to be stuck into a bone is in a columnar form having a conical tip and is provided with two to six ridge portions disposed parallel to the axis of the columnar portion thereof and having a length extending within the bone.

And, the present inventor has found the following matters. That is, it is preferred that the head portion of the fixing pin is in a disc-like form having a diameter of 2 to 5 mm and a thickness of 0.3 to 1.0 mm; that the columnar portion of the thumbtack shaft portion has a diameter of 0.6 to 1.0 mm and a length of 1.5 to 5.5 mm; and that the ridge portions have each a width of 0.05 to 0.3 mm and a height of 5 to 30 μm. Also, it is preferred that the ridge portions have a uniform lateral cross-sectional shape over a substantially full length thereof; and that the tip of each ridge portion in an opposite side to the head portion of the fixing pin is in a convergent form.

### BRIEF DESCRIPTION OF THE DRAWINGS

FIG. 1 is a perspective view of one embodiment of a fixing pin of a dental tissue regeneration membrane according to the present invention as seen from the obliquely upper position;

FIG. 2 is a perspective view of the fixing pin of the dental tissue regeneration membrane of FIG. 1 as seen from the obliquely lower position; and

FIG. 3 is a front view of a fixing pin of a dental tissue regeneration membrane of the conventional art.

### DESCRIPTION OF THE PREFERRED EMBODIMENTS

The fixing pin of the dental tissue regeneration membrane according to the present invention will be described below in detail with reference to the drawings.

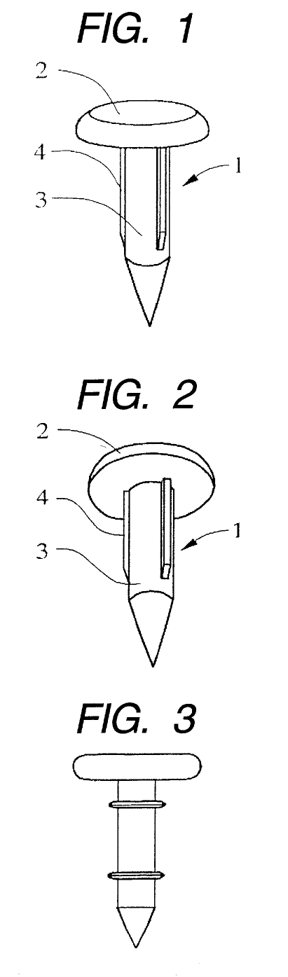

FIG. 1 is a perspective view of one embodiment of a fixing pin of a dental tissue regeneration membrane according to the present invention as seen from the obliquely upper position; FIG. 2 is a perspective view of the fixing pin of the dental tissue regeneration membrane of FIG. 1 as seen from the obliquely lower position; and FIG. 3 is a front view of a fixing pin of a dental tissue regeneration membrane of the conventional art.

In the drawings, a numeral 1 is a fixing pin of a dental tissue regeneration membrane according to the present invention, wherein a periphery in an oral cavity inner surface side of a head portion 2 supporting a dental guided tissue regeneration membrane is at least in a smooth curve surface form; and a thumbtack shaft portion 3 to be stuck into a bone is in a columnar form having a conical tip and is provided with two to six ridge portions 4 disposed parallel to the axis of the columnar portion of the thumbtack shaft portion 3 and having a length extending within the bone.

Preferably, the head portion 2 is as small as possible so far as it can support the dental guided tissue regeneration membrane and is in a disc-like form having a diameter of 2 to 5 mm and a thickness of 0.3 to 1.0 mm and having a periphery in a smooth curve surface form in an oral cavity inner surface side thereof. The head portion 2 may be in a round head form like as a cut-off of a part of the spherical surface. The reason why the periphery of the head portion 2 in the oral cavity inner surface side thereof is made at least in a smooth curve surface form resides in avoiding the head portion 2 to give stimulation to a gingival flap covering an operated portion and to injure it. At this time, it is preferred that to provide the portion in a smooth curve surface form does not reduce the strength of the periphery of the head portion 2. Accordingly, in the case where the head portion 2 is in a disc-like form having a constant thickness, it is preferred that the curve surface has a radius the same as the thickness.

The thumbtack shaft portion 3 is stuck into a prepared hole of a bone in a site for the fixing pin 1 to be stuck, having been bored previously by means of a drill, and having a diameter substantially the same as the diameter of the columnar portion of the thumbtack shaft portion 3, thereby supporting the dental guided tissue regeneration membrane by the head portion 2. Accordingly, it is preferred that the thumbtack shaft portion 3 has a conical portion at the tip thereof such that it easily sticks, and has a columnar portion having a diameter of 0.6 to 1.0 mm and a length of 1.5 to 5.5 mm such that it comes into contact with the bone of the prepared hole over a length to some extent.

When the fixing pin 1 is stuck into the prepared hole having been previously bored by means of a drill in the site of the bone into which the fixing pin 1 is to be stuck, and having a diameter substantially the same as the diameter of the columnar potion of the thumbtack shaft portion 3, the ridge portions 4 increase a contact area with the bone of the prepared hole, thereby preventing the fixing pin 1 from dropping out from the prepared hole as well as increasing the stiffness of the thumbtack shaft portion 3. However, since it is not desired that the resistance during sticking the fixing pin 1 into the prepared hole is high, two to six of the ridge portions 4 are provided parallel to the axis of the thumbtack shaft portion 3. When the number of the ridge portions 4 is less than two, the coming-out preventing action is not obtained sufficiently. On the other hand, when the number of the ridge portions 4 exceeds six, the resistance during sticking the thumbtack shaft portion 3 is too high, and therefore, such is not proper. As the lateral cross-sectional shape of the ridge portions 4, employable are various shapes including not only a quadrangular shape in the embodiment as shown in the drawings but also a triangular shape, a semicircular shape, and a quadrangular shape having an arc portion provided in the tip thereof. Further, while the lateral cross-sectional shape of the ridge portions 4 in the direction parallel to the axis of the thumbtack shaft portion 3 are generally uniform over a substantially full length thereof, it may be convergent towards the tip of the thumbtack shaft portion 3. In any of these cases, it is preferred that the tip of each of the ridge portions 4 in the opposite side to the head portion 2 is in a convergent form. And, it is preferred that the lateral cross-sectional shape of each of the ridge portions 4 has a size of 0.05 to 0.3 mm in width and 5 to 30 μm in height.

The fixing pin of the dental tissue regeneration membrane according to the present invention is made of a bioabsorbable material and is prepared by, for example, heating and softening a homopolymer or copolymer, as a polymeric material, of, e.g., L-lactic acid, DL-lactic acid, glycolic acid, and ε-polycaprolactone, followed by injection molding.

The fixing pin 1 of the dental tissue regeneration membrane according to the present invention is fixed to a bone in the same manner as in the conventional fixing pins of dental tissue regeneration membrane. That is, a dental guided tissue regeneration membrane is positioned in a site where a periodontal tissue is to be subjected to regeneration treatment; a hole having a diameter substantially the same as the thickness of the thumbtack shaft portion 3 of the fixing pin 1 of the dental tissue regeneration membrane is previously bored for preparation in an appropriate place of a bone into which the fixing pin 1 of the dental tissue regeneration membrane is to be stuck; and the fixing pin 1 of the dental tissue regeneration membrane is then stuck into the prepared hole.

During this operation, the ridge portions 4 provided in the thumbtack shaft portion 3 acts not only to increase the cross-sectional area of the thumbtack shaft portion 3 but also to increase the stiffness of the thumbtack shaft portion 3. And, when the fixing pin 1 has been once stuck into the bone, the ridge portions generate a force in the direction expanding the bone to the surrounding thereof to the prepared hole provided in the bone, whereby the fixing pin 1 of the dental tissue regeneration membrane is strongly held within the bone to achieve a dropping-out preventing effect.

(Embodiment)

Actually, there was manufactured on trial the fixing pin 1 of the dental tissue regeneration membrane made of poly-L-lactic acid (a molecular weight: 300,000) and constructed of the head portion 2 having a diameter of 2.5 mm and a thickness of 0.5 mm and the thumbtack shaft portion 3 comprising the columnar portion having a diameter of 0.8 mm and a length of 2.0 mm and two of the ridge portions 4 provided in opposite positions on the thumbtack shaft portion 3 and having a quadrangular lateral cross-sectional shape of 0.1 mm in width and 10 μm in height. The fixing pin 1 of the dental tissue regeneration membrane was inserted into a Duracon-made block having been previously provided with a prepared hole having a diameter of 0.8 mm, and the assembly was subjected to a drawing test using a rheometer (a trade name: CR-200, manufactured by Sun Kagaku Co., Ltd.). As a result, the fixing pin 1 of the dental tissue regeneration membrane showed a drawing strength of 562 g, and therefore, it could be confirmed that the drawing strength greatly increased as compared with the drawing strength of 483 g in a fixing pin not provided with the ridge portion 4.

Incidentally, since the fixing pin 1 of the dental tissue regeneration membrane according to the present invention is made of a bioabsorbable material, it is decomposed and absorbed within a living body after regeneration of the periodontal tissue, likewise bioabsorbable dental tissue regeneration membranes. Accordingly, as a matter of course, it is not necessary to remove the fixing pin 1 of the dental tissue regeneration membrane according to the present invention. Further, in the actual clinical field, as a matter of course, the fixing pin 1 of the dental tissue regeneration membrane according to the present invention can be employed in combination with a method for binding and fixing it to a tooth with a suture, or a method for sandwiching and fixing it between an implant main body and a cover screw.

As described above in detail, the fixing pin of the dental tissue regeneration membrane according to the present invention is a fixing pin which during fixing a dental guided tissue regeneration membrane, can be smoothly stuck into a prepared hole bored in a bone without being broken, and after being once stuck, does not readily drop out but can surely fix the dental guided tissue regeneration membrane. Further, since the fixing pin of the dental tissue regeneration membrane according to the present invention is decomposed and absorbed within a living body after a periodontal tissue has been regenerated, it is not required to carry out a re-operation for removing it, and therefore, is not burdensome to a patient and an operator. In consequence, the invention greatly contributes to the dental remedy.

While the invention has been described in detail and with reference to specific embodiments thereof, it will be apparent to one skilled in the art that various changes and modifications can be made therein without departing from the spirit and scope thereof.

What is claimed is:

1. A fixing pin of a dental tissue regeneration membrane comprising a bioabsorbable material for fixing a dental guided tissue regeneration membrane, wherein:
a periphery in an oral cavity inner surface side of a head portion thereof supporting a dental guided tissue regeneration membrane is at least in a smooth curve surface form; and
a thumbtack shaft portion thereof to be stuck into a bone is in a columnar form having a conical tip and is provided with two to six ridge portions disposed parallel to the axis of the columnar portion thereof and having a length extending within the bone.

2. The fixing pin of the dental tissue regeneration membrane as claimed in claim 1, wherein:
the head portion of the fixing pin is in a disc-like form having a diameter of 2 to 5 mm and a thickness of 0.3 to 1.0 mm;
the columnar portion of the thumbtack shaft portion has a diameter of 0.6 to 1.0 mm and a length of 1.5 to 5.5 mm; and
the ridge portions have each a width of 0.05 to 0.3 mm and a height of 5 to 30 μm.

3. The fixing pin of the dental tissue regeneration membrane as claimed in claim 1 or 2, wherein:
the ridge portions have a uiform lateral cross-sectional shape over a substantially full length thereof; and
the tip of each ridge portion in an opposite side to the head portion of the fixing pin is in a convergent form.
